(19) United States
(12) Patent Application Publication
(10) Pub. No.: US 2026/0177517 A1
(43) Pub. Date: Jun. 25, 2026

(54) SENSOR AND METHOD FOR MANUFACTURING THE SAME

(71) Applicant: KABUSHIKI KAISHA TOSHIBA, Tokyo (JP)

(72) Inventors: Shihe WANG, Kawasaki Kanagawa (JP); Yosuke AKIMOTO, Yokohama Kanagawa (JP); Hiroshi HAMASAKI, Hiratsuka Kanagawa (JP); Yui YAMAZAKI, Ota Tokyo (JP)

(73) Assignee: KABUSHIKI KAISHA TOSHIBA, Tokyo (JP)

(21) Appl. No.: 19/257,583

(22) Filed: Jul. 2, 2025

(30) Foreign Application Priority Data
Dec. 19, 2024 (JP) 2024-223717

(51) Int. Cl. G01N 27/12; G01N 33/00

(52) U.S. Cl. CPC G01N 27/12; G01N 33/0027

(57) ABSTRACT

According to one embodiment, a sensor includes a first base, a first detection section, and a second detection section. The first detection section includes first fixed portions fixed to the first base, first connecting portions, and a first element. The second detection section includes second fixed portions fixed to the first base, second connecting portions, and a second element. One of the second connecting portions is supported by one of the second fixed portions. The second element includes a second resistive member. The second element includes a second inner portion and a second outer portion. The second outer portion is around the second inner portion. The second inner portion is fixed to the first base. A second distance between the first base and the second outer portion is less likely to change than a first distance between the first base and the first element.

### CROSS-REFERENCE TO RELATED APPLICATIONS

This application is based upon and claims the benefit of priority from Japanese Patent Application No.2024-223717, filed on Dec. 19, 2024; the entire contents of which are incorporated herein by reference.

### FIELD

Embodiments described herein relate generally to a sensor and a method for manufacturing the same.

### BACKGROUND

For example, there is a sensor using a MEMS (Micro Electro Mechanical Systems) element, etc. It is desirable to improve the characteristics of the sensor.

### BRIEF DESCRIPTION OF THE DRAWINGS

FIGS. 1A and 1B are schematic views illustrating a sensor according to a first embodiment;

FIGS. 2A and 2B are schematic views illustrating a sensor according to the first embodiment;

FIGS. 3A and 3B are schematic views illustrating a sensor according to the first embodiment;

FIGS. 4A and 4B are schematic views illustrating a sensor according to the first embodiment;

FIGS. 5A and 5B are schematic views illustrating a sensor according to the first embodiment;

FIGS. 6A and 6B are schematic views illustrating a part of a sensor according to a second embodiment;

FIGS. 7A and 7B are schematic views illustrating a sensor according to a third embodiment; and

FIG. 8 is a flow chart illustrating a method for manufacturing a sensor according to a fourth embodiment.

### DETAILED DESCRIPTION

According to one embodiment, a sensor includes a first base, a first detection section, and a second detection section. The first detection section includes a plurality of first fixed portions fixed to the first base, a plurality of first connecting portions, and a first element. One of the plurality of first connecting portions is supported by one of the plurality of first fixed portions. The first element is supported by the plurality of first connecting portions and includes a first resistive member. A first gap is provided between the first base and the first element. The second detection section includes a plurality of second fixed portions fixed to the first base, a plurality of second connecting portions, and a second element. One of the plurality of second connecting portions is supported by one of the plurality of second fixed portions. The second element includes a second resistive member. The second element includes a second inner portion and a second outer portion. The second outer portion is around the second inner portion. The second outer portion is supported by the plurality of second connecting portions. The second inner portion is fixed to the first base. A second gap is provided between the first base and the second outer portion. A second distance between the first base and the second outer portion is less likely to change than a first distance between the first base and the first element.

Various embodiments are described below with reference to the accompanying drawings.

The drawings are schematic and conceptual; and the relationships between the thickness and width of portions, the proportions of sizes among portions, etc., are not necessarily the same as the actual values. The dimensions and proportions may be illustrated differently among drawings, even for identical portions.

In the specification and drawings, components similar to those described previously or illustrated in an antecedent drawing are marked with like reference numerals, and a detailed description is omitted as appropriate.

### First Embodiment

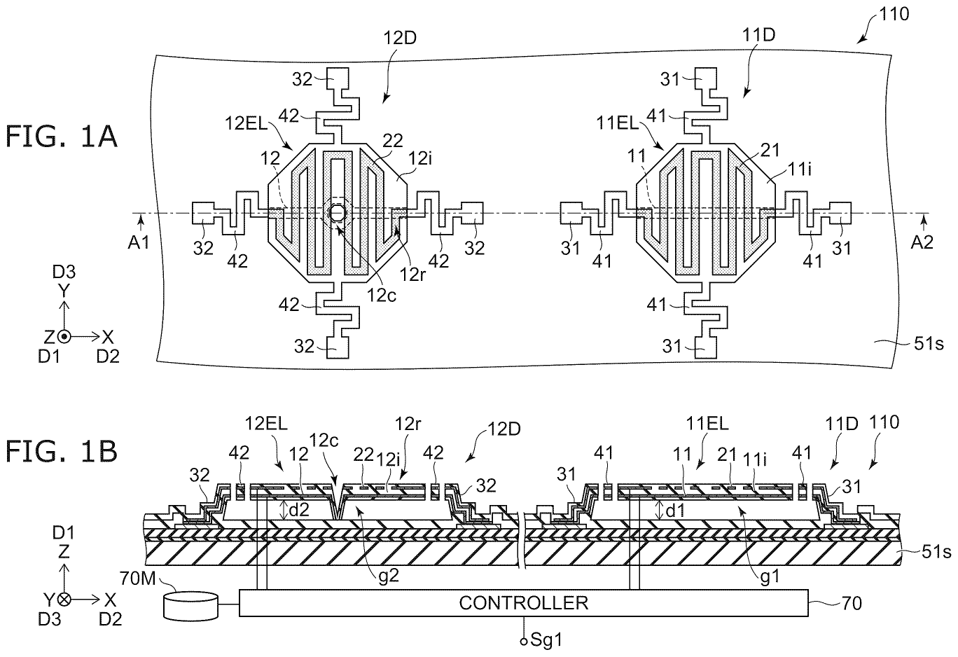

FIGS. 1A and 1B are schematic views illustrating a sensor according to a first embodiment.

FIG. 1B is a cross-sectional view taken along the line A1-A2 in FIG. 1A.

As shown in FIGS. 1A and 1B, a sensor 110 according to the embodiment includes a first base 51s, a first detection section 11D, and a second detection section 12D.

The first detection section 11D includes a plurality of first fixed portions 31, a plurality of first connecting portions 41, and a first element 11EL. The plurality of first fixed portions 31 are fixed to the first base 51s.

A direction from the first base 51s to the plurality of first fixed portions 31 is defined as a first direction D1. The first direction D1 is defined as a Z-axis direction. One direction perpendicular to the Z-axis direction is defined as an X-axis direction. A direction perpendicular to the Z-axis direction and the X-axis direction is defined as a Y-axis direction.

The first base 51s is, for example, along the X-Y plane.

The plurality of first connecting portions 41 are each supported by the plurality of first fixed portions 31. For example, one of the plurality of first connecting portions 41 is supported by one of the plurality of first fixed portions 31.

The first element 11EL is supported by the plurality of first connecting portions 41. The first element 11EL includes a first resistive member 11. The first element 11EL may further include a first conductive member 21. A first gap g1 is provided between the first base 51s and the first element 11EL. The first element 11EL may be in the form of a film along the X-Y plane.

The second detection section 12D includes a plurality of second fixed portions 32, a plurality of second connecting portions 42, and a second element 12EL. The plurality of second fixed portions 32 are fixed to the first base 51s. The plurality of second connecting portions 42 are each supported by the plurality of second fixed portions 32. For example, one of the plurality of second connecting portions 42 is supported by one of the plurality of second fixed portions 32.

The second element 12EL is supported by the plurality of second connecting portions 42. The second element 12EL includes a second resistive member 12. The second element 12EL may further include a second conductive member 22.

The second element 12EL includes a second inner portion 12c and a second outer portion 12r. The second outer portion 12r is around the second inner portion 12c. The second outer portion 12r may correspond to an outer edge of the second element 12EL. The second inner portion 12c may correspond to a center of the second element 12EL. The second outer portion 12r is supported by the plurality of second connecting portions 42. The second inner portion 12c is fixed to the first base 51s. A second gap g2 is provided between the first base 51s and the second outer portion 12r.

The outer edge of the first element 11EL is supported by the plurality of first connecting portions 41. Meanwhile, in the second element 12EL, the second outer portion 12r of the second element 12EL is supported by the plurality of second connecting portions 42, and the second inner portion 12c of the second element 12EL is fixed to the first base 51s. Thereby, the distance between the first base 51s and the second element 12EL is less likely to change than the distance between the first base 51s and the first element 11EL. The distance between the first base 51s and the second element 12EL does not substantially change.

In the embodiment, the second distance d2 between the first base 51s and the second outer portion 12r is less likely to change than the first distance d1 between the first base 51s and the first element 11EL.

For example, a first electrical resistance R1 of the first resistive member 11 is configured to change in response to a detection target around the first detection section 11D and the second detection section 12D. A second electrical resistance R2 of the second resistive member 12 is configured to change in response to the detection target.

For example, the first electrical resistance R1 of the first resistive member 11 changes depending on a state of the detection target. The detection target can be detected by detecting the first electrical resistance R1. The second electrical resistance R2 of the second resistive member 12 changes depending on the state of the detection target. The detection target can be detected by detecting the second electrical resistance R2. The detection target may be, for example, a gas (such as carbon dioxide). The sensor 110 is, for example, a gas sensor.

For example, a value based on the first electrical resistance R1 (for example, the first detection result) may be corrected by a value based on the second electrical resistance R2 (for example, the second detection result).

For example, the first distance d1 between the first base 51s and the first element 11EL may change due to a change in temperature of the first element 11EL. For example, the first distance d1 between the first base 51s and the first element 11EL may change due to changes over time. When the first distance d1 between the first base 51s and the first element 11EL changes, the thermal characteristics (e.g., heat dissipation) of the first element 11EL change. This may cause the first electrical resistance R1 to change unintentionally, separate from the state of the object to be detected.

On the other hand, the change in the second distance d2 between the first base 51s and the second element 12EL (second outer portion 12r) is small. The change in the second electrical resistance R2 due to the change in the second distance d2 is small. The value based on the first electrical resistance R1 is corrected using a value based on the second electrical resistance R2 being detected, so that the detection target can be detected more accurately. According to the embodiment, a sensor capable of improving characteristics can be provided.

As shown in FIG. 1B, a controller 70 may be provided in the sensor 110. The controller 70 is configured to perform a first operation of outputting an output signal Sg1. The output signal Sg1 is obtained by a second operation of correcting a first value based on the first electrical resistance R1 based on a second value based on the second electrical resistance R2. In the second operation, for example, correction (or calibration) is performed. By the first operation being performed based on the second operation, a more accurate detection result can be obtained. The controller 70 may include a processor.

The controller 70 may be configured to repeatedly perform the first operation and the second operation plurality of times. Calibration may be performed periodically.

In the embodiment, a coefficient relating to the relationship between the first electrical resistance R1 obtained from the first resistive member 11 and the concentration of the detection target may be corrected by the second value (e.g., the second detection result) based on the second electrical resistance R2. For example, the detection result of the first electrical resistance R1 is converted to the concentration of the detection target (e.g., the gas concentration) based on the coefficient. The converted value (concentration) corresponds to the value based on the first electrical resistance R1. In this case as well, the detection target can be accurately detected by correcting the coefficient by the second value (e.g., the second detection result) based on the second electrical resistance R2.

As shown in FIGS. 1A and 1B, the first element 11EL may further include the first conductive member 21. The second element 12EL may further include the second conductive member 22. The controller 70 may be configured to supply a first power to the first conductive member 21 when the first electrical resistance R1 is detected. The controller 70 may be configured to supply a second power to the second conductive member 22 when the second electrical resistance R2 is detected. These conductive members are, for example, heaters. The temperature when the electrical resistance is detected changes depending on the state of the object to be detected.

As shown in FIGS. 1A and 1B, the first element 11EL may include a first insulating member 11i. At least a part of the first insulating member 11i is provided between the first resistive member 11 and the first conductive member 21. The second element 12EL may include a second insulating member 12i. At least a part of the second insulating member 12i is provided between the second resistive member 12 and the second conductive member 22.

A wiring connected to the first resistive member 11 may be electrically connected to the controller 70 via any of the plurality of first connecting portions 41. A wiring connected to the first conductive member 21 may be electrically connected to the controller 70 via any of the plurality of first connecting portions 41. The wiring connected to the second resistive member 12 may be electrically connected to the controller 70 via any of the plurality of second connecting portions 42. The wiring connected to the second conductive member 22 may be electrically connected to the controller 70 via any of the plurality of second connecting portions 42.

As shown in FIG. 1A, a length (first length) of one of the plurality of first connecting portions 41 may be longer than a width (first width) of the one of the plurality of first connecting portions 41. The first length is a length of the one of the plurality of first connecting portions 41 along a first extending direction in which the one of the plurality of first connecting portions 41 extends. The first width is a length of the one of the plurality of first connecting portions 41 along a first crossing direction that crosses the first extending direction. Each of the plurality of first connecting portions 41 may have, for example, a bent structure. Each of the plurality of first connecting portions 41 may have, for example, a meandering structure. Such a structure suppresses heat dissipation from the first element 11EL. Stable characteristics are easy to obtain.

As shown in FIG. 1A, a length (second length) of one of the plurality of second connecting portions 42 may be longer than a width (second width) of the one of the plurality of second connecting portions 42. The second length is a length of the one of the plurality of second connecting portions 42 along a second extending direction in which the one of the plurality of second connecting portions 42 extends. The second width is a length of one of the plurality of second connecting portions 42 along the second crossing direction that crosses the second extending direction. Each of the plurality of second connecting portions 42 may have, for example, a bent structure. Each of the plurality of second connecting portions 42 may have, for example, a meandering structure. Such a structure suppresses heat dissipation from the second element 12EL. Stable characteristics are easy to obtain.

As shown in FIG. 1B, a first memory 70M may be provided in the sensor 110. The first memory 70M may be configured to store detection results. The first memory 70M may be configured to store values (such as coefficients) used in at least one of the first operation or the second operation described above. The controller 70 may be configured to control the operation of the first memory 70M. The controller 70 may be configured to store information acquired from outside in the first memory 70M.

In the sensor 110, for example, in a second direction D2, the first element 11EL is located between one of the plurality of first connecting portions 41 and another one of the plurality of first connecting portions 41. The second direction D2 crosses the first direction D1. The second direction D2 may be, for example, the X-axis direction. For example, in a third direction D3, the first element 11EL is located between one of the plurality of first connecting portions 41 and another one of the plurality of first connecting portions 41. The third direction D3 crosses a plane including the first direction D1 and the second direction D2. The third direction D3 may be, for example, the Y-axis direction.

In the sensor 110, for example, in the second direction D2, the second element 12EL is located between one of the plurality of second connecting portions 42 and another one of the plurality of second connecting portions 42. For example, in the third direction D3, the second element 12EL is located between one of the plurality of second connecting portions 42 and another one of the plurality of second connecting portions 42.

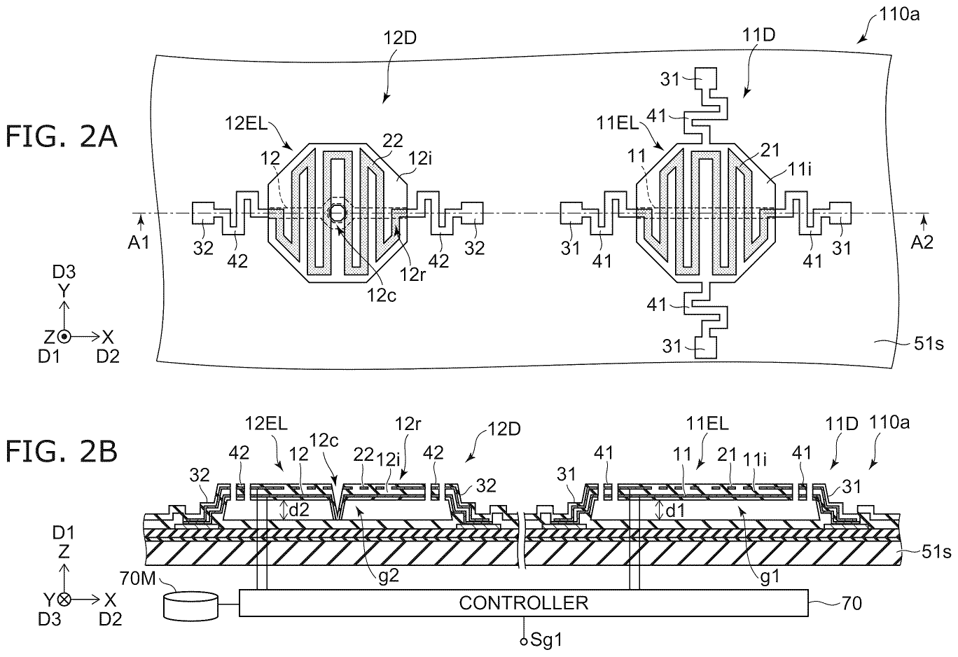

FIGS. 2A and 2B are schematic views illustrating a sensor according to the first embodiment.

FIG. 2B is a cross-sectional view taken along the line A1-A2 in FIG. 2A.

As shown in FIGS. 2A and 2B, in a sensor 110a according to the embodiment, the configuration of the second detection section 12D is different from the configuration of the first detection section 11D. The configuration of the sensor 110a except for this may be the same as the configuration of the sensor 110.

In the sensor 110a, a number of second connecting portions 42 (second number) is different from a number of first connecting portions 41 (first number). In this example, the second number is smaller than the first number. The second connecting portions 42 becomes heat dissipation paths for the second element 12EL. The heat dissipation through the connection member in the second element 12EL is different from the heat dissipation through the connection member in the first element 11EL.

As already explained, the second distance d2 is less likely to change than the first distance d1. In the first element 11EL and the second element 12EL, a difference in heat dissipation to the first base 51s is provided due to the difference in distance. Furthermore, as described above, a difference in heat dissipation via connecting members may also be provided. For example, the second operation may be performed to correct the first value based on the first electrical resistance R1 based on the second value based on the second electrical resistance R2.

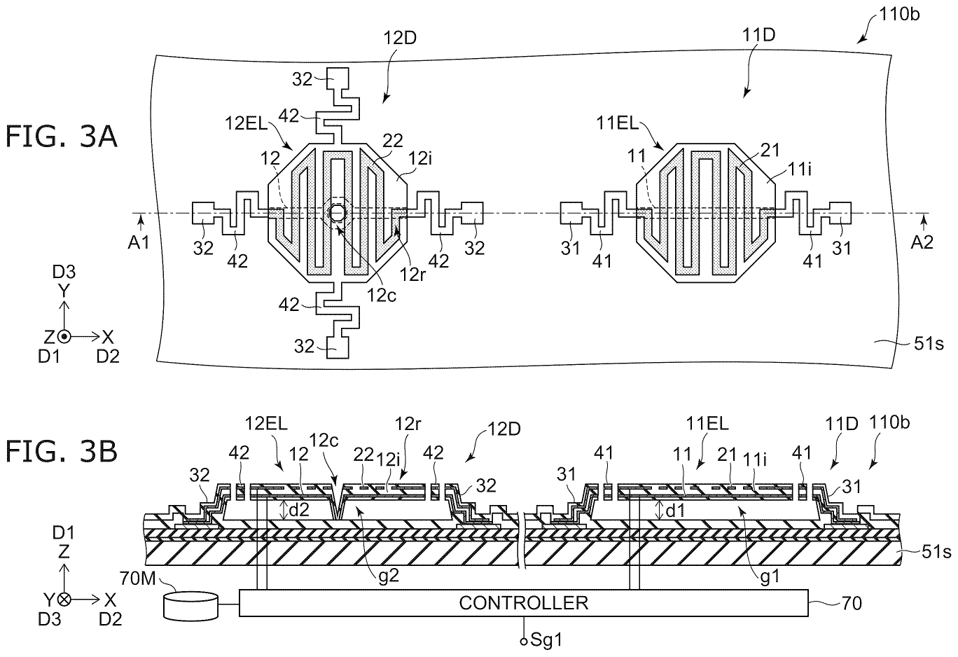

FIGS. 3A and 3B are schematic views illustrating a sensor according to the first embodiment.

FIG. 3B is a cross-sectional view taken along the line A1-A2 in FIG. 3A.

As shown in FIGS. 3A and 3B, in a sensor 110b according to the embodiment, the configuration of the second detection section 12D is different from the configuration of the first detection section 11D. The configuration of the sensor 110b except for this may be the same as the configuration of the sensor 110. In sensor 110b, the second number is greater than the first number. In the embodiment, the second number may be smaller than the first number.

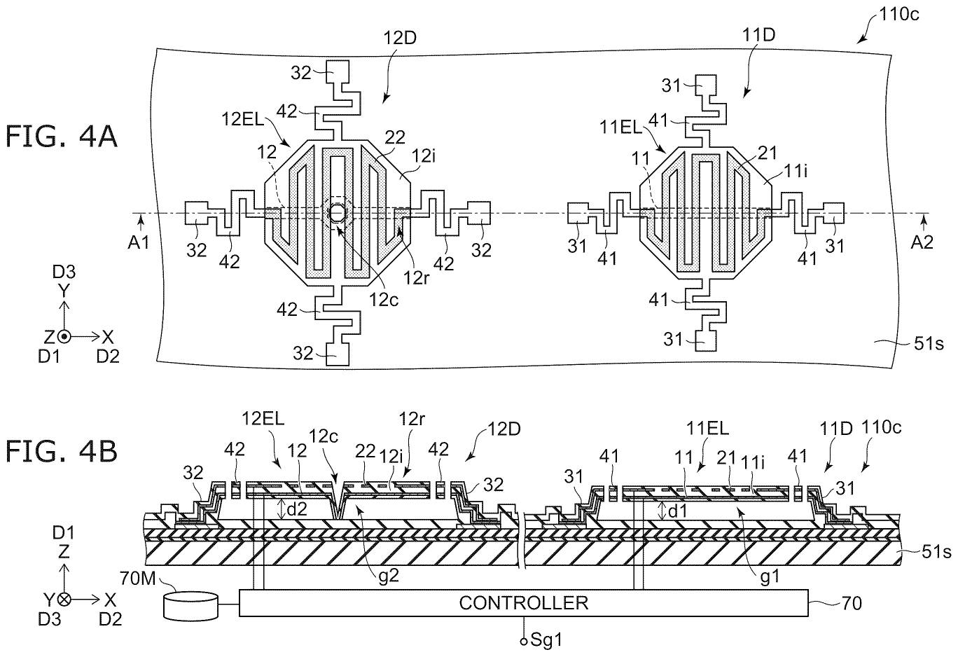

FIGS. 4A and 4B are schematic views illustrating a sensor according to the first embodiment.

FIG. 4B is a cross-sectional view taken along the line A1-A2 in FIG. 4A.

As shown in FIGS. 4A and 4B, in a sensor 110c according to the embodiment, the configuration of the second detection section 12D is different from the configuration of the first detection section 11D. The configuration of the sensor 110c except for this may be the same as the configuration of the sensor 110, the sensor 110a or the sensor 110b.

In the sensor 110c, an area of the second element 12EL (second area) is different from an area of the first element 11EL (first area). In this example, the second area is larger than the first area. The difference in area causes a difference in heat dissipation.

In the first element 11EL and the second element 12EL, a difference in heat dissipation to the first base 51s due to the difference between the first distance d1 and the second distance d2, and a difference in heat dissipation due to the difference in area may be provided. For example, the second operation may be performed to correct the first value based on the first electrical resistance R1 based on the second value based on the second electrical resistance R2.

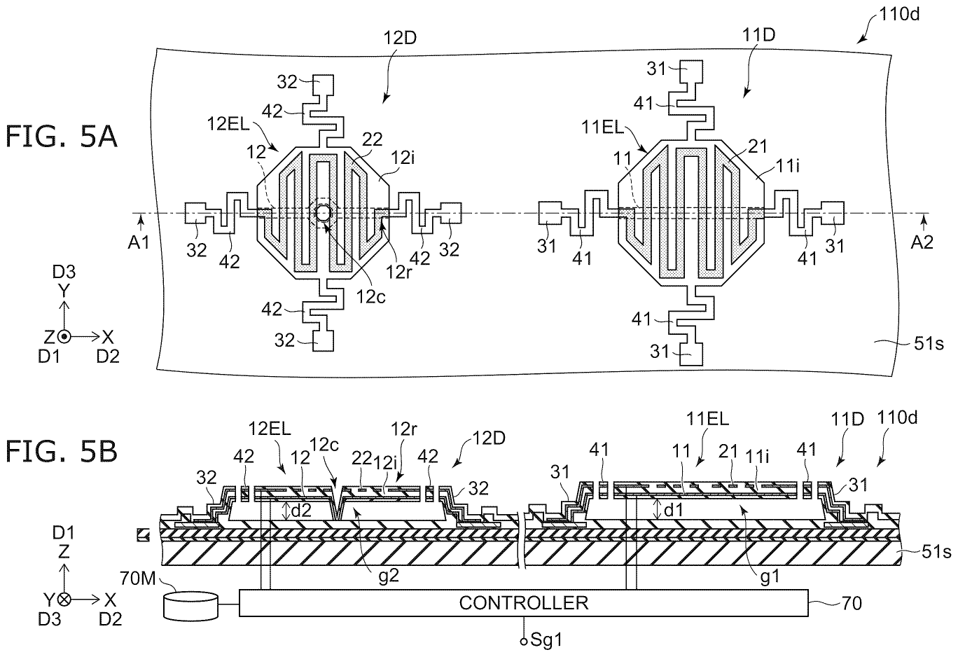

FIGS. 5A and 5B are schematic views illustrating a sensor according to the first embodiment.

FIG. 5B is a cross-sectional view taken along the line A1-A2 in FIG. 5A.

As shown in FIGS. 5A and 5B, in a sensor 110d according to the embodiment, the configuration of the second detection section 12D is different from the configuration of the first detection section 11D. The configuration of the sensor 110d except for this may be the same as the configuration of the sensor 110. In the sensor 110d, the second area is smaller than the first area. In the embodiment, the second area may be smaller than the first area.

As described above, in the embodiment, the first detection section 11D and the second detection section 12D may satisfy at least one of a first condition or a second condition. In the first condition, the second number of the plurality of second connecting portions 42 is different from the first number of the plurality of first connecting portions 41. In the second condition, the second area of the second element 12EL is different from the first area of the first element 11EL.

### Second Embodiment

In a second embodiment, the sensor further includes a third detection section 13D in addition to the first detection section 11D and the second detection section 12D described above. In the second embodiment, the first detection section 11D and the second detection section 12D may have the configuration described in relation to the first embodiment. Below, an example of the third detection section 13D is described.

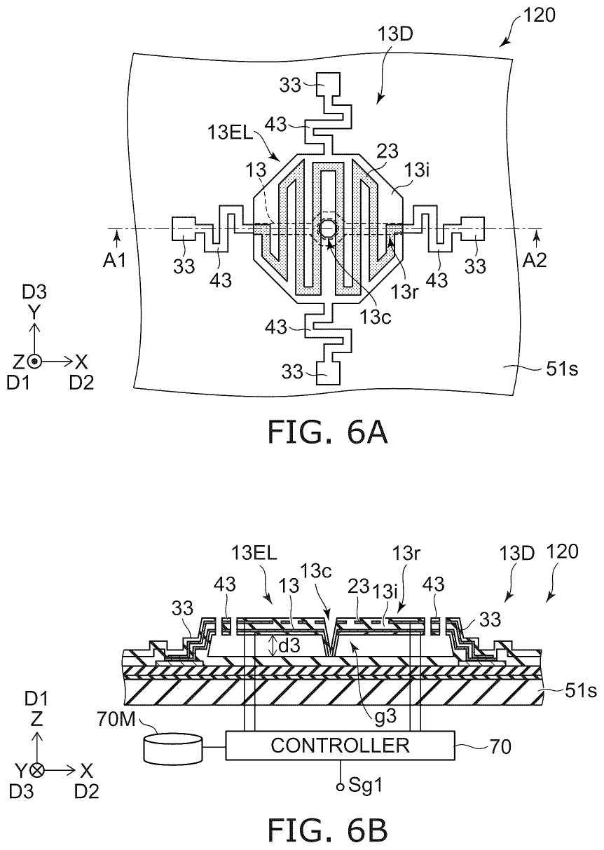

FIGS. 6A and 6B are schematic views illustrating a part of a sensor according to the second embodiment.

FIG. 6B is a cross-sectional view taken along the line A1-A2 in FIG. 6A.

As shown in FIGS. 6A and 6B, a sensor 120 according to the embodiment includes a third detection section 13D. The sensor 120 includes the first detection section 11D and second detection section 12D described in relation to the first embodiment.

The third detection section 13D includes a plurality of third fixed portions 33, a plurality of third connecting portions 43, and a third element 13EL. The plurality of third fixed portions 33 are fixed to the first base 51s. The plurality of third connecting portions 43 are each supported by the plurality of third fixed portions 33. For example, one of the plurality of third connecting portions 43 is supported by one of the plurality of third fixed portions 33.

The third element 13EL is supported by the plurality of third connecting portions 43. The third element 13EL includes a third resistive member 13. The third element 13EL may further include a third conductive member 23.

The third element 13EL includes a third inner portion 13c and a third outer portion 13r. The third outer portion 13r is around the third inner portion 13c. The third outer portion 13r may correspond to an outer edge of the third element 13EL. The third inner portion 13c may correspond to a center of the third element 13EL. The third outer portion 13r is supported by the plurality of third connecting portions 43. The third inner portion 13c is fixed to the first base 51s.

A third gap g3 is provided between the first base 51s and the third outer portion 13r. A third distance d3 between the first base 51s and the third outer portion 13r is less likely to change than the first distance d1.

The first electrical resistance R1 of the first resistive member 11 is configured to change in response to the detection target around the first detection section 11D, the second detection section 12D, and the third detection section 13D. The second electrical resistance R2 of the second resistive member 12 is configured to change in response to the detection target. A third electrical resistance R3 of the third resistive member 13 is configured to change in response to the detection target.

In the embodiment, the configuration of the third elements 13EL may be different from the configuration of the second elements 12EL. For example, a third area of a plurality of the third elements 13EL is different from a second area of a plurality of the second elements 12EL.

For example, a difference in heat dissipation is provided between the third element 13EL and the second element 12EL due to the difference in area. By using the two detection results obtained from these two elements, the state of the detection target can be detected more accurately.

For example, the value (detection result) based on the first electrical resistance R1 may be corrected based on the second electrical resistance R2 and the third resistive member 13. For example, the controller 70 configured to perform the first operation of outputting the output signal Sg1 may be provided. The output signal Sg1 is obtained by the second operation of correcting the first value (first detection result) based on the first electrical resistance R1 based on at least one of the second value (second detection result) based on the second electrical resistance R2 or a third value (third detection result) based on the third electrical resistance R3. Such correction allows the detection target to be detected more accurately.

The controller 70 may be configured to repeatedly perform the first operation and the second operation plurality of times. For example, the correction (or calibration) may be performed periodically. For example, the obtained correction coefficients may be stored in the first memory 70M or the like.

The third detection section 13D described with reference to FIGS. 6A and 6B may be combined with the sensor 110a. In this case, a number of the plurality of third connecting portions 43 (the third number) is different from the number of the plurality of second connecting portions 42 (the second number). For example, the third number is greater than the second number. A difference in heat dissipation may be provided between the second element 12EL and the third element 13EL based on the number of connecting portions.

Thus, the second detection section 12D and the third detection section 13D may satisfy at least one of a third condition or a fourth condition. In the third condition, the third number of the plurality of third connecting portions 43 is different from the second number of the plurality of second connecting portions 42. In the fourth condition, the third area of the plurality of third elements 13EL is different from the second area of the plurality of second elements 12EL.

In the sensor 120, the first element 11EL may include the first conductive member 21. The second element 12EL may include the second conductive member 22. The third element 13EL may include the third conductive member 23. The controller 70 may be configured to supply the first power to the first conductive member 21 when the first electrical resistance R1 is detected. The controller 70 may be configured to supply the second power to the second conductive member 22 when the second electrical resistance R2 is detected. The controller 70 may be configured to supply a third power to the third conductive member 23 when the third electrical resistance R3 is detected.

As shown in FIG. 6A, a length (third length) of one of the plurality of third connecting portions 43 may be longer than a width (third width) of the one of the plurality of third connecting portions 43. The third length is a length of the one of the plurality of third connecting portions 43 along a third extending direction in which the one of the plurality of third connecting portions 43 extends. The third width is a length of the one of the plurality of third connecting portions 43 along a third crossing direction that crosses the third extending direction. Each of the plurality of third connecting portions 43 may have, for example, a bent structure. Each of the plurality of third connecting portions 43 may have, for example, a meandering structure. Such a structure suppresses heat dissipation from the third element 13EL. Stable characteristics are easy to obtain.

As shown in FIGS. 6A and 6B, the third element 13EL may include a third insulating member 13i. At least a part of the third insulating member 13i is provided between the third resistive member 13 and the third conductive member 23.

### Third Embodiment

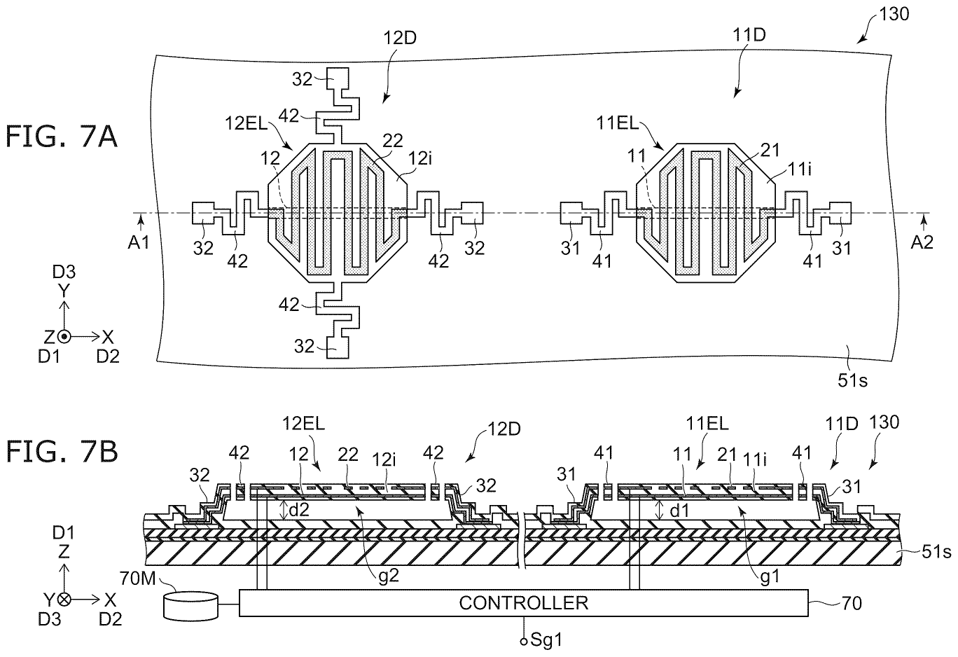

FIGS. 7A and 7B are schematic views illustrating a sensor according to a third embodiment.

FIG. 7B is a cross-sectional view taken along the line A1-A2 in FIG. 7A.

As shown in FIGS. 7A and 7B, a sensor 130 according to the embodiment includes the first base 51s, the first detection section 11D, and the second detection section 12D.

The first detection section 11D includes the plurality of first fixed portions 31, the plurality of first connecting portions 41, and the first element 11EL. The plurality of first fixed portions 31 are fixed to the first base 51s. The plurality of first connecting portions 41 are each supported by the plurality of first fixed portions 31. For example, one of the plurality of first connecting portions 41 is supported by one of the plurality of first fixed portions 31. The first element 11EL is supported by the plurality of first connecting portions 41. A first gap g1 is provided between the first base 51s and the first element 11EL.

The second detection section 12D includes the plurality of second fixed portions 32, the plurality of second connecting portions 42, and the second element 12EL. The plurality of second fixed portions 32 are fixed to the first base 51s. The plurality of second connecting portions 42 are each supported by the plurality of second fixed portions 32. For example, one of the plurality of second connecting portions 42 is supported by one of the plurality of second fixed portions 32. The second element 12EL is supported by the plurality of second connecting portions 42. The second element 12EL includes the second resistive member 12. A second gap g2 is provided between the first base 51s and the second element 12EL.

In the sensor 130, the number (second number) of the plurality of second connecting portions 42 is different from the number (first number) of the plurality of first connecting portions 41.

By the difference in the number of connecting portions, a difference in heat dissipation is provided. By using the detection signals obtained from these two detection sections, the detection target can be detected more accurately.

As already explained, the first electrical resistance R1 of the first resistive member 11 is configured to change in response to the detection target around the first detection section 11D and the second detection section 12D. The second electrical resistance R2 of the second resistive member 12 is configured to change in response to the detection target.

The sensor 130 may be provided with the controller 70. The controller 70 is configured to perform the first operation of outputting the output signal Sg1. The output signal Sg1 is obtained by the second operation of correcting the first value (detection result) based on the first electrical resistance R1 based on the second value based on the second electrical resistance R2.

In the sensor 130, the second distance d2 between the first base 51s and the second element 12EL may be less likely to change than the first distance d1 between the first base 51s and the first element 11EL.

### Fourth Embodiment

The method for manufacturing a sensor according to the fourth embodiment is a method for manufacturing a sensor (such as sensor 110) that includes the first base 51s, the first detection section 11D, the first memory 70M, and the controller 70.

As already explained, the first detection section 11D includes the plurality of first fixed portions 31 fixed to the first base 51s, the plurality of first connecting portions 41, and the first element 11EL. One of the plurality of first connecting portions 41 is supported by one of the plurality of first fixed portions 31. The first element 11EL is supported by the plurality of first connecting portions 41. A first gap g1 is provided between the first base 51s and the first element 11EL.

The controller 70 is configured to correct a first value based on the first electrical resistance R1 of the first resistive member 11, based on a first information stored in the first memory 70M.

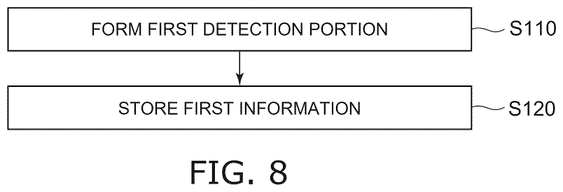

FIG. 8 is a flow chart illustrating a method for manufacturing a sensor according to the fourth embodiment.

As shown in FIG. 8, the manufacturing method according to the embodiment includes forming the first detection section 11D (step S110). The manufacturing method includes storing the first information in the first memory 70M (step S120).

At least a part of the first information is obtained from the second detection section 12D. The second detection section 12D may include the plurality of second fixed portions 32, the plurality of second connecting portions 42, and the second element 12EL. The plurality of second fixed portions 32 are fixed to a base (e.g., a second base) separate from the first base 51s. One of the plurality of second connecting portions 42 is supported by one of the plurality of second fixed portions 32. The second element 12EL is supported by the plurality of second connecting portions 42. The second element 12EL includes the second resistive member 12. The second element 12EL may further include the second conductive member 22.

The second element 12EL includes the second inner portion 12c and the second outer portion 12r. The second outer portion 12r is provided around the second inner portion 12c. The second outer portion 12r is supported by the plurality of second connecting portions 42. The second inner portion 12c is fixed to the second base (see FIG. 1B, etc.). A second gap g2 is provided between the second base and the second outer portion 12r. For example, the second distance d2 between the second base and the second outer portion 12r is less likely to change than the first distance d1 between the first base 51s and the first element 11EL.

In the method for manufacturing the sensor according to the embodiment, information derived from the second value (detection result) based on the second electrical resistance R2 obtained from the second element 12EL of the second detection section 12D as described above is stored in the first memory 70M. The first information includes, for example, a correction coefficient. By the first information being stored in the first memory 70M, correction is performed in the controller 70. According to the embodiment, a method for manufacturing a sensor capable of improving characteristics is provided.

The second base described above may correspond to a part of the first base 51s exemplified in FIGS. 1A and 1B, etc.

In the manufacturing method according to the embodiment, a part of the first information may further be obtained from the third detection section 13D. The third detection section 13D may include the plurality of third fixed portions 33 fixed to the second base, the plurality of third connecting portions 43, and the third element 13EL. One of the plurality of third connecting portions 43 is supported by one of the plurality of third fixed portions 33. The third element 13EL is supported by the plurality of third connecting portions 43.

The third element 13EL includes the third inner portion 13c and the third outer portion 13r. The third outer portion 13r is provided around the third inner portion 13c. The third outer portion 13r is supported by the plurality of third connecting portions 43. The third inner portion 13c is fixed to the second base. A third gap g3 is provided between the second base and the third outer portion 13r.

The third distance d3 between the second base and the third outer portion 13r is less likely to change than the first distance d1. For example, the second detection section 12D and the third detection section 13D satisfy at least one of the third condition or the fourth condition. In the third condition, the third number of the plurality of third connecting portions 43 is different from the second number of the plurality of second connecting portions 42. In the fourth condition, the third area of the plurality of third elements 13EL is different from the second area of the plurality of second elements 12EL. A manufacturing method for a sensor capable of improving characteristics is provided.

The embodiment may include the following Technical proposals:

### (Technical Proposal 1)

A sensor, comprising:
a first base;
a first detection section; and
a second detection section,
the first detection section including:
   a plurality of first fixed portions fixed to the first base;
   a plurality of first connecting portions, one of the plurality of first connecting portions being supported by one of the plurality of first fixed portions; and
   a first element supported by the plurality of first connecting portions and including a first resistive member,
a first gap being provided between the first base and the first element,
the second detection section including:
   a plurality of second fixed portions fixed to the first base;
   a plurality of second connecting portions, one of the plurality of second connecting portions being supported by one of the plurality of second fixed portions; and
   a second element including a second resistive member, the second element including a second inner portion and a second outer portion, the second outer portion being around the second inner portion, the second outer portion being supported by the plurality of second connecting portions, and the second inner portion being fixed to the first base,
a second gap being provided between the first base and the second outer portion, and
a second distance between the first base and the second outer portion being less likely to change than a first distance between the first base and the first element.

### (Technical Proposal 2)

The sensor according to Technical proposal 1, wherein

a first length of the one of the plurality of first connecting portions is longer than a first width of the one of the plurality of first connecting portions,

the first length is a length of the one of the plurality of first connecting portions along a first extending direction in which the one of the plurality of first connecting portions extends, and

the first width is a length of the one of the plurality of first connecting portions along a first crossing direction that crosses the first extending direction.

### (Technical Proposal 3)

The sensor according to Technical proposal 1 or 2, wherein
a first electrical resistance of the first resistive member is configured to change in response to a detection target around the first detection section and the second detection section, and
a second electrical resistance of the second resistive member is configured to change in response to the detection target.

### (Technical Proposal 4)

The sensor according to Technical proposal 3, further comprising:
a controller configured to perform a first operation of outputting an output signal,
the output signal being obtained by a second operation of correcting a first value based on the first electrical resistance based on a second value based on the second electrical resistance.

### (Technical Proposal 5)

The sensor according to Technical proposal 4, wherein
the first element further includes a first conductive member,
the second element further includes a second conductive member,
the controller is configured to supply a first power to the first conductive member when the first electrical resistance is detected, and
the controller is configured to supply a second power to the second conductive member when the second electrical resistance is detected.

### (Technical Proposal 6)

The sensor according to Technical proposals 4 or 5, wherein
the controller is configured to repeatedly perform the first operation and the second operation plurality of times.

### (Technical Proposal 7)

The sensor according to any one of Technical proposals 1-6, wherein
the first detection section and the second detection section satisfy at least one of a first condition or a second condition,
in the first condition, a second number of the plurality of second connecting portions is different from a first number of the plurality of first connecting portions, and
in the second condition, a second area of the second element is different from a first area of the first element.

### (Technical Proposal 8)

The sensor according to Technical proposal 1 or 2, further comprising:
a third detection section,
the third detection section including:
   a plurality of third fixed portions fixed to the first base;
   a plurality of third connecting portions, one of the plurality of third connecting portions being supported by one of the plurality of third fixed portions; and
   a third element including a third resistive member, the third element including a third inner portion and a third outer portion, the third outer portion being around the third inner portion, the third outer portion being supported by the plurality of third connecting portions, and the third inner portion being fixed to the first base,
a third gap being provided between the first base and the third outer portion, and
a third distance between the first base and the third outer portion being less likely to change than the first distance.

### (Technical Proposal 9)

The sensor according to Technical proposal 8, wherein
a third number of the plurality of third connecting portions is different from a second number of the plurality of second connecting portions.

### (Technical Proposal 10)

The sensor according to Technical proposal 8, wherein
a third area of the plurality of third elements is different from a second area of the plurality of second elements.

### (Technical Proposal 11)

The sensor according to Technical proposal 8, wherein
a first electrical resistance of the first resistive member is configured to change in response to a detection target around the first detection section, the second detection section, and the third detection section,
a second electrical resistance of the second resistive member is configured to change in response to the detection target, and
a third electrical resistance of the third resistive member is configured to change in response to the detection target.

### (Technical Proposal 12)

The sensor according to Technical proposal 11, further comprising:
a controller configured to perform a first operation of outputting an output signal,
the output signal being obtained by a second operation of correcting a first value based on the first electrical resistance based on at least one of a second value based on the second electrical resistance or a third value based on the third electrical resistance.

### (Technical Proposal 13)

The sensor according to Technical proposal 12, wherein
the controller is configured to repeatedly perform the first operation and the second operation plurality of times.

### (Technical Proposal 14)

The sensor according to Technical proposal 12 or 13, wherein
the first element further includes a first conductive member,
the second element further includes a second conductive member,
the third element further includes a third conductive member,
the controller is configured to supply a first power to the first conductive member when the first electrical resistance is detected,
the controller is configured to supply a second power to the second conductive member when the second electrical resistance is detected, and
the controller is configured to supply a third power to the third conductive member when the third electrical resistance is detected.

### (Technical Proposal 15)

A sensor, comprising:
a first base;
a first detection section; and
a second detection section,
the first detection section including:
   a plurality of first fixed portions fixed to the first base;
   a plurality of first connecting portions, one of the plurality of first connecting portions being supported by one of the plurality of first fixed portions; and
   a first element supported by the plurality of first connecting portions and including a first resistive member,
a first gap being provided between the first base and the first element,
the second detection section including:
   a plurality of second fixed portions fixed to the first base;
   a plurality of second connecting portions, one of the plurality of second connecting portions being supported by one of the plurality of second fixed portions; and
   a second element supported by the plurality of second connecting portions and including a second resistive member,
a second gap being provided between the first base and the second element, and
a second number of the plurality of second connecting portions being different from a first number of the plurality of first connecting portions.

### (Technical Proposal 16)

The sensor according to Technical proposal 15, wherein
a second distance between the first base and the second element is less likely to change than a first distance between the first base and the first element.

### (Technical Proposal 17)

The sensor according to Technical proposal 15 or 16, wherein
a first electrical resistance of the first resistive member is configured to change in response to a detection target around the first detection section and the second detection section, and
a second electrical resistance of the second resistive member being configured to change in response to the detection target.

### (Technical Proposal 18)

The sensor according to Technical proposal 17, further comprising:
a controller configured to perform a first operation of outputting an output signal,
the output signal being obtained by a second operation of correcting a first value based on the first electrical resistance based on a second value based on the second electrical resistance.

### (Technical Proposal 19)

A method for manufacturing a sensor, the sensor including a first base, a first detection section, a first memory, and a controller,
the first detection section including:
   a plurality of first fixed portions fixed to the first base;
   a plurality of first connecting portions, one of the plurality of first connecting portions being supported by one of the plurality of first fixed portions; and
   a first element supported by the plurality of first connecting portions and including a first resistive member,
a first gap being provided between the first base and the first element,
the controller being configured to correct a first value based on a first electrical resistance of the first resistive member based on first information stored in the first memory,
the manufacturing method including:
   forming the first detection section; and
   storing the first information in the first memory,
at least a part of the first information being obtained from the second detection section,
the second detection section including:
   a plurality of second fixed portions fixed to a second base;
   a plurality of second connecting portions, one of the plurality of second connecting portions being supported by one of the plurality of second fixed portions; and
   a second element including a second resistive member, the second element including a second inner portion and a second outer portion, the second outer portion being around the second inner portion, the second outer portion being supported by the plurality of second connecting portions, the second inner portion being fixed to the second base,
a second gap being provided between the second base and the second outer portion, and
a second distance between the second base and the second outer portion being less likely to change than a first distance between the first base and the first element.

### (Technical Proposal 20)

The method for manufacturing the sensor according to Technical Proposal 19, wherein
a part of the first information is further obtained from a third detection section,
the third detection section includes:
   a plurality of third fixed portions fixed to the second base;
   a plurality of third connecting portion, one of the plurality of third connecting portions being supported by one of the plurality of third fixed portions; and
a third element including a third resistive member, the third element including a third inner portion and a third outer portion, the third outer portion being around the third inner portion, the third outer portion being supported by the plurality of third connecting portions, and the third inner portion being fixed to the second base,
a third gap is provided between the second base and the third outer portion,
a third distance between the second base and the third outer portion is less likely to change than the first distance,
the second detection section and the third detection section satisfy at least one of a third condition or a fourth condition,
in the third condition, a third number of the third connection portions is different from a second number of the second connection portions, and
in the fourth condition, a third area of the third elements is different from a second area of the second elements.

According to the embodiment, a sensor capable of improving characteristics and a method for manufacturing the same can be provided.

Hereinabove, exemplary embodiments of the invention are described with reference to specific examples. However, the embodiments of the invention are not limited to these specific examples. For example, one skilled in the art may similarly practice the invention by appropriately selecting specific configurations of components included in sensors such as bases, detection sections, controllers, etc., from known art. Such practice is included in the scope of the invention to the extent that similar effects thereto are obtained.

Further, any two or more components of the specific examples may be combined within the extent of technical feasibility and are included in the scope of the invention to the extent that the purport of the invention is included.

Moreover, all sensors and all methods for manufacturing the same practicable by an appropriate design modification by one skilled in the art based on the sensors and the methods for manufacturing the same described above as embodiments of the invention also are within the scope of the invention to the extent that the purport of the invention is included.

Various other variations and modifications can be conceived by those skilled in the art within the spirit of the invention, and it is understood that such variations and modifications are also encompassed within the scope of the invention.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention.

What is claimed is:

1. A sensor, comprising:
a first base;
a first detection section; and
a second detection section,
the first detection section including:
a plurality of first fixed portions fixed to the first base;
a plurality of first connecting portions, one of the plurality of first connecting portions being supported by one of the plurality of first fixed portions; and
a first element supported by the plurality of first connecting portions and including a first resistive member,
a first gap being provided between the first base and the first element,
the second detection section including:
a plurality of second fixed portions fixed to the first base;
a plurality of second connecting portions, one of the plurality of second connecting portions being supported by one of the plurality of second fixed portions; and
a second element including a second resistive member, the second element including a second inner portion and a second outer portion, the second outer portion being around the second inner portion, the second outer portion being supported by the plurality of second connecting portions, and the second inner portion being fixed to the first base,
a second gap being provided between the first base and the second outer portion, and
a second distance between the first base and the second outer portion being less likely to change than a first distance between the first base and the first element.

2. The sensor according to claim 1, wherein
a first length of the one of the plurality of first connecting portions is longer than a first width of the one of the plurality of first connecting portions,
the first length is a length of the one of the plurality of first connecting portions along a first extending direction in which the one of the plurality of first connecting portions extends, and
the first width is a length of the one of the plurality of first connecting portions along a first crossing direction that crosses the first extending direction.

3. The sensor according to claim 1, wherein
a first electrical resistance of the first resistive member is configured to change in response to a detection target around the first detection section and the second detection section, and
a second electrical resistance of the second resistive member is configured to change in response to the detection target.

4. The sensor according to claim 3, further comprising:
a controller configured to perform a first operation of outputting an output signal,
the output signal being obtained by a second operation of correcting a first value based on the first electrical resistance based on a second value based on the second electrical resistance.

5. The sensor according to claim 4, wherein
the first element further includes a first conductive member,
the second element further includes a second conductive member,
the controller is configured to supply a first power to the first conductive member when the first electrical resistance is detected, and
the controller is configured to supply a second power to the second conductive member when the second electrical resistance is detected.

6. The sensor according to claim 4, wherein
the controller is configured to repeatedly perform the first operation and the second operation plurality of times.

7. The sensor according to claim 1, wherein
the first detection section and the second detection section satisfy at least one of a first condition or a second condition,
in the first condition, a second number of the plurality of second connecting portions is different from a first number of the plurality of first connecting portions, and
in the second condition, a second area of the second element is different from a first area of the first element.

8. The sensor according to claim 1, further comprising:
a third detection section,
the third detection section including:
a plurality of third fixed portions fixed to the first base;
a plurality of third connecting portions, one of the plurality of third connecting portions being supported by one of the plurality of third fixed portions; and
a third element including a third resistive member, the third element including a third inner portion and a third outer portion, the third outer portion being around the third inner portion, the third outer portion being supported by the plurality of third connecting portions, and the third inner portion being fixed to the first base,
a third gap being provided between the first base and the third outer portion, and
a third distance between the first base and the third outer portion being less likely to change than the first distance.

9. The sensor according to claim 8, wherein
a third number of the plurality of third connecting portions is different from a second number of the plurality of second connecting portions.

10. The sensor according to claim 8, wherein
a third area of the plurality of third elements is different from a second area of the plurality of second elements.

11. The sensor according to claim 8, wherein
a first electrical resistance of the first resistive member is configured to change in response to a detection target around the first detection section, the second detection section, and the third detection section,
a second electrical resistance of the second resistive member is configured to change in response to the detection target, and
a third electrical resistance of the third resistive member is configured to change in response to the detection target.

12. The sensor according to claim 11, further comprising:
a controller configured to perform a first operation of outputting an output signal,
the output signal being obtained by a second operation of correcting a first value based on the first electrical resistance based on at least one of a second value based on the second electrical resistance or a third value based on the third electrical resistance.

13. The sensor according to claim 12, wherein
the controller is configured to repeatedly perform the first operation and the second operation plurality of times.

14. The sensor according to claim 12, wherein
the first element further includes a first conductive member,
the second element further includes a second conductive member,
the third element further includes a third conductive member,
the controller is configured to supply a first power to the first conductive member when the first electrical resistance is detected,
the controller is configured to supply a second power to the second conductive member when the second electrical resistance is detected, and
the controller is configured to supply a third power to the third conductive member when the third electrical resistance is detected.

15. A sensor, comprising:
a first base;
a first detection section; and
a second detection section,
the first detection section including:
a plurality of first fixed portions fixed to the first base;
a plurality of first connecting portions, one of the plurality of first connecting portions being supported by one of the plurality of first fixed portions; and
a first element supported by the plurality of first connecting portions and including a first resistive member,
a first gap being provided between the first base and the first element,
the second detection section including:
a plurality of second fixed portions fixed to the first base;
a plurality of second connecting portions, one of the plurality of second connecting portions being supported by one of the plurality of second fixed portions; and
a second element supported by the plurality of second connecting portions and including a second resistive member,
a second gap being provided between the first base and the second element, and
a second number of the plurality of second connecting portions being different from a first number of the plurality of first connecting portions.

16. The sensor according to claim 15, wherein
a second distance between the first base and the second element is less likely to change than a first distance between the first base and the first element.

17. The sensor according to claim 15, wherein
a first electrical resistance of the first resistive member is configured to change in response to a detection target around the first detection section and the second detection section, and
a second electrical resistance of the second resistive member being configured to change in response to the detection target.

18. The sensor according to claim 17, further comprising:
a controller configured to perform a first operation of outputting an output signal,
the output signal being obtained by a second operation of correcting a first value based on the first electrical resistance based on a second value based on the second electrical resistance.

19. A method for manufacturing a sensor, the sensor including a first base, a first detection section, a first memory, and a controller,
the first detection section including:
a plurality of first fixed portions fixed to the first base;
a plurality of first connecting portions, one of the plurality of first connecting portions being supported by one of the plurality of first fixed portions; and
a first element supported by the plurality of first connecting portions and including a first resistive member,
a first gap being provided between the first base and the first element,
the controller being configured to correct a first value based on a first electrical resistance of the first resistive member based on first information stored in the first memory,
the manufacturing method including:
forming the first detection section; and
storing the first information in the first memory,
at least a part of the first information being obtained from the second detection section,
the second detection section including:
a plurality of second fixed portions fixed to a second base;
a plurality of second connecting portions, one of the plurality of second connecting portions being supported by one of the plurality of second fixed portions; and
a second element including a second resistive member, the second element including a second inner portion and a second outer portion, the second outer portion being around the second inner portion, the second outer portion being supported by the plurality of second connecting portions, the second inner portions being fixed to the second base,
a second gap being provided between the second base and the second outer portion, and
a second distance between the second base and the second outer portion being less likely to change than a first distance between the first base and the first element.

20. The method for manufacturing the sensor according to claim 19, wherein
a part of the first information is further obtained from a third detection section,
the third detection section includes:
a plurality of third fixed portions fixed to the second base;
a plurality of third connecting portion, one of the plurality of third connecting portions being supported by one of the plurality of third fixed portions; and
a third element including a third resistive member, the third element including a third inner portion and a third outer portion, the third outer portion being around the third inner portion, the third outer portion being supported by the plurality of third connecting portions, and the third inner portion being fixed to the second base,
a third gap is provided between the second base and the third outer portion,
a third distance between the second base and the third outer portion is less likely to change than the first distance,
the second detection section and the third detection section satisfy at least one of a third condition or a fourth condition,
in the third condition, a third number of the third connection portions is different from a second number of the second connection portions, and
in the fourth condition, a third area of the third elements is different from a second area of the second elements.
